(19) United States
(12) Patent Application Publication
(10) Pub. No.: US 2021/0154462 A1
(43) Pub. Date: May 27, 2021

(54) DISINFECTING CAP WITH SIDE OPENING

(71) Applicant: DRMA GROUP INTERNATIONAL LLC, Palm City, FL (US)

(72) Inventor: Zoran Drmanovic, Palm City, FL (US)

(21) Appl. No.: 16/693,262

(22) Filed: Nov. 23, 2019

(51) Int. Cl. A61M 39/20; A61M 39/16; A61L 2/26; A61L 2/16

(52) U.S. Cl. CPC A61M 39/20; A61M 39/162; A61M 2039/1072; A61L 2/26; A61L 2/16; A61M 39/165

(57) ABSTRACT

A capping device for disinfecting a medical implement includes a first portion having a bottom opening and a first inner surface, a second portion having a medial opening, a second inner surface, and a disinfecting member attached to the second inner surface, and a connector coupling the first and second portions in a manner which permits movement of the second portion between a closed position wherein the second portion is in contact with the first portion so as to form a hollow defined by the first inner surface and the second inner surface, and an open position wherein the second portion is apart from the first portion. The capping device is configured for attachment to the medical implement through the bottom opening to bring an access portion of the medical implement in contact with the disinfecting member when the capping device is in the closed position.

### BACKGROUND

The present invention generally relates to a device having disinfecting properties, and more specifically, to a capping device for disinfecting a hub or injection port.

Presence of intravenous catheters is the major risk factor for the development of bloodstream infections. These catheters can be either central or peripheral. Needleless hub connectors are ordinarily used as an injection port on the catheters. A typical connector includes a female luer lock, and usually, a syringe with a male luer lock is used to inject fluids or medications.

Needleless hub connector contamination is a major risk factor for bloodstream infection. Scrubbing the female luer lock with alcohol for 10-60 seconds is recommended before each use, but this procedure is often omitted by medical professionals. Studies have shown better results in reducing infection with different types of alcohol impregnated protectors, such as Swab Cap® by ICU Medical or Curos® by 3M. However, because the protector constitutes a separate entity, only the most diligent medical professionals would utilize them after every step. In addition, the cap does not assure mandatory compliance. Caps do not always engage the threads on the hub, and the threads can serve as a source of infection, especially, if a dirty or bloody male luer from the syringe is used to engage the hub. Also, because of their small size, the caps are easily lost or contaminated after use if they are placed on a contaminated surface. The medical professional may forget to put it back, place dirty cap back onto the hub or need to use a new clean cap. Those caps also require two hands to attach or detach the cap from the hub while the present invention requires provider to use only one hand to hold and open the top of the invention while the other one is used to connect syringe and inject.

Attempts have been made to cover the hub in order to keep it disinfected. However, these efforts either failed to cover the hub completely by shielding only the top membrane, or were too difficult to remove when the port needed to be injected quickly and conveniently.

Thus, there remains a need for a convenient and reliable disinfecting device that would stay at the top of needleless hub all the time, would guarantee 100% compliance of medical professionals with antiseptic techniques and at the same time be very easy to open in order to connect with a syringe.

### SUMMARY

An embodiment provides a device for disinfecting a medical implement, including:
a first portion having a bottom opening and a first inner surface,
a second portion having a medial opening, a second inner surface, and a disinfecting member attached to the second inner surface, and
a connector coupling the first portion of the capping device and the second portion thereof in a manner which permits movement of the second portion between
   a closed position wherein the second portion of the capping device is in contact with the first portion thereof so as to form a hollow defined by the first inner surface and the second inner surface, and
   an open position wherein the second portion is apart from the first portion,
wherein the capping device is configured for attachment to the medical implement through the bottom opening to bring an access portion of the medical implement in contact with the disinfecting member when the capping device is in the closed position.

The first portion may include a base having the bottom opening and a side angled to the base, wherein the side may include a medial opening.

The side may be permanently attached to the base at an angle of about 90°.

The side may be configured to extend above the access portion of the medical implement.

The side may be permanently attached to base, wherein the medial opening of the side is configured to face the medical implement.

The medial opening of the second portion may be configured to face the medical implement.

The second portion may include a sealing member separating the medial opening of the second portion into a top chamber and a bottom chamber.

The disinfecting member may be disposed in the top chamber of the second portion of the capping device.

The sealing member may include an elevated central rim.

The disinfecting member may be disposed over an entire inner surface of the top chamber.

The top chamber may further include a hollow disposed between the disinfecting member and the medial opening.

The shape of the hollow may substantially match the shape of a portion of the medical implement to be disinfected.

The second portion may further include a handle disposed on an external surface thereof.

The second portion may separate from the first portion by application of a force to the handle.

The disinfecting member may be impregnated with a disinfecting agent.

When the disinfecting member is in contact with the access portion of the medical implement, the disinfecting member may release the disinfecting agent onto the portion to be disinfected.

The medical implement may be a needleless hub or an injection port.

The access portion of the medical implement may include a female luer fitting and an injection membrane.

The access portion of the medical implement in the open position may be completely uncovered.

The access portion of the medical implement in the closed position may be completely covered with a disinfecting member.

Yet another embodiment provides a method for disinfecting a medical implement, including:
providing a device including:
a first portion having a bottom opening and a first inner surface,
a second portion having a medial opening, a second inner surface, and a disinfecting member attached to the second inner surface, and
a connector coupling the first portion of the capping device and the second portion thereof in a manner which permits movement of the second portion between
   a closed position wherein the second portion of the capping device is in contact with the first portion thereof so as to form a hollow defined by the first inner surface and the second inner surface, and
   an open position wherein the second portion is apart from the first portion,
wherein the capping device is configured for attachment to the medical implement through the bottom opening to bring an access portion of the medical implement in contact with the disinfecting member when the capping device is in the closed position,
attaching the device to an access portion of the medical implement through the bottom opening to bring the disinfecting member in contact with the access portion;
displacing the second portion from the closed position to the open position to expose the access portion of the medical implement; and
attaching a source of fluid to the medical implement.

The method may further include:
injecting the fluid into the medical implement;
detaching the source of fluid from the medical implement; and
displacing the second portion from the open position to the closed position to bring the disinfecting member back in contact with the portion to be disinfected.

### BRIEF DESCRIPTION OF THE DRAWINGS

The above and other aspects and features of the present disclosure will become more apparent by describing in detail exemplary embodiments thereof with reference to the attached drawings, in which:

FIG. 1 is a view of an embodiment of the capping device completely detached from a medical implement in closed position;

FIG. 2 is a cross sectional view of an embodiment of the capping device attached to a medical implement in closed position;

FIG. 3 is a prospective view of an embodiment of the capping device attached to a medical implement in open position;

FIG. 4 is a prospective view of moving/flexible side of the device;

FIG. 5 is a perspective view of a fixed side of the embodiment of the capping device attached to a medical implement;

FIG. 6 is a prospective view of the disinfecting pad according to an embodiment;

FIG. 7 is a partial view of the disinfecting pad according to an embodiment showing only its top portion; and

FIG. 8 is a partial view of the disinfecting pad according to an embodiment showing its main body.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below by referring to the figures to explain aspects of the present disclosure. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third, etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross-section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“Substantially” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “substantially” can mean within one or more standard deviations, or within ±30%, 20%, 10%, 5% of the stated value.

Attention is now drawn to FIGS. 1-4 depicting embodiments of the present invention. FIG. 1 shows an embodiment of a capping device 100 completely detached from a medical implement in closed position. The capping device 100 includes a first portion 101 including a bottom opening 115 and a first inner surface 104, a second portion 108 including a medial opening, a second inner surface 109, and a disinfecting member 116 attached to the second inner surface 109. The disinfecting member 116 may be impregnated with a disinfecting agent, which may be any disinfecting agent known in the art.

The capping device may further include a connector 110 coupling the first portion 101 of the capping device and the second portion 108 thereof in a manner which permits movement of the second portion 108 between a closed position (shown in FIG. 1), wherein the second portion 108 of the capping device is in contact with the first portion 101 thereof so as to form a hollow defined by the first inner surface 104 and the second inner surface 109, and an open position (shown in FIG. 3), wherein the second portion 108 is apart from the first portion 101. The capping device 100 is configured for attachment to a medical implement through the bottom opening 115 to bring an access portion of the medical implement in contact with the disinfecting member 116 when the capping device is in the closed position.

The size of the second portion 108 may be the same or slightly smaller than the size of the first portion 101 so the second portion 108 may enter inside the first portion 101 when the device is closed (shown in FIG. 1). However, the overlap between the first portion 101 and the second portion 108 is not necessary when the contacting surfaces of the first and second portions match well and close securely, for example, by using a slip-on mechanism.

The capping device and the medical implement may be permanently affixed to each other during a manufacturing process so as to form a single entity. In another embodiment, the capping device and the medical implement may not be permanently affixed. This latter embodiment allows the disinfecting apparatus to be added to any connection hub on any desired intravenous line. The medical professional may thereby attach the disinfecting apparatus to a hub of his or her choice, including the hub that he or she frequently uses. The portion to be disinfected may be a needleless hub or an injection port. The needleless hub may include female luer threads 120 and an injection membrane 119.

At the bottom of the device is the bottom opening 115 where the top portion of the medical implement containing the female luer threads 120 and the injection membrane 119 enters. The bottom opening 115 may be covered by a protecting member (not shown), which may for example be a peelable foil. The protecting member serves as a fluid barrier preventing loss of the disinfecting member 116 before the capping device is used for the first time. The protecting member may also provide a visible indication that the seal is broken. A medical professional would need to remove the protecting member prior to connecting the apparatus with the medical implement.

The bottom opening 115 may be surrounded by an edge 114 to facilitate attachment of the capping device 100 to the medical implement. The edge 114 may be round and may have a diameter similar to the diameter of the neck of the most common medical implements. The inner surface of the edge 114 may be covered with an adhesive material which is revealed after the protective member is removed. This adhesive material may help provide a better connection between the edge 114 and the access portion of the medical implement. It also may also contribute to the prevention of leakage of the disinfecting agent when the sealing member (described below) fails to create a perfect seal. The diameter of the bottom opening 115 matches well with the diameter of the access portion of the medical implement, so the two could potentially connect, for example, via a slip lock without a need for the adhesive to keep the capping device and the medical implement together.

The second portion 108 may include a handle 111 or upward protrusions in order to facilitate a lateral displacement of the second portion 108 that includes the disinfecting member 116 to the open position, wherein the portion of the medical implement to be disinfected may be accessed for injection. The shape and size of the handle would allow for easy lateral displacement and may include some ridges on its surface for easy gripping.

The housing may me made of a polyalkylene material (such as polyethylene or polypropylene), a polyester material, a polyurethane material, a silicone material, rubber, a cotton material, or any combination thereof.

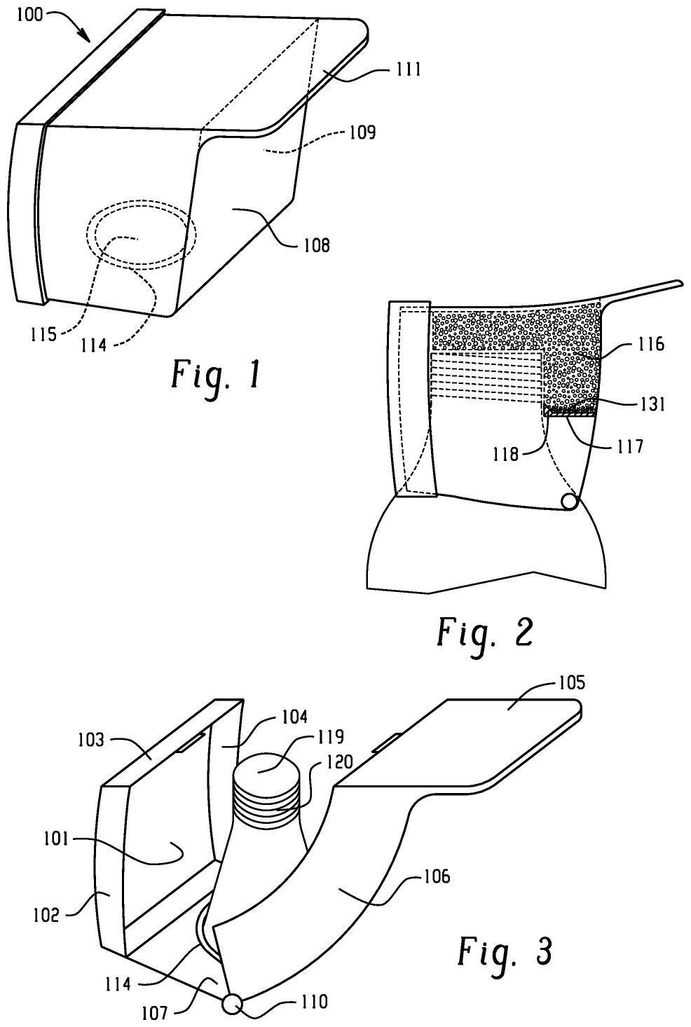

FIG. 2 is a cross-sectional view of an embodiment attached to a medical implement in a closed position. The figure shows the first portion 101 and the second portion 108 to which the handle 111 is attached. The second portion has a medial opening and can be divided into a top chamber containing the disinfecting member 116 and the bottom chamber which may be hollow to allow easy displacement of the second portion 108 from the closed position to the open position. The second portion 108 may further include a sealing member 117 which separates the top and bottom chambers and provides a good seal with the access portion of the medical implement in order to minimize the loss of the disinfecting agent when the device is open for the medical implement to connect to the syringe.

FIG. 2 further provides a cross-section view of the disinfecting member 116 in the closed position, where it directly contacts and completely covers the injection membrane 119 of the medical implement. The disinfecting member 116 may also be in direct contact with the female luer threads 120. In the open position (shown in FIG. 3), the access portion of the medical implement may be completely uncovered. The disinfecting member 116 may be impregnated with a disinfecting agent 131 which may be present on the sealing member 117. The connector 110 may be a hinge which may bias the first portion 101 and the second portion 108 to close the capping device 100. The disinfecting member 116 may have excellent absorption capacity, so when pressure is applied to push the second portion 108 of the capping device to the closed position to cover the access portion of the medical implement, the disinfecting member 116 may undergo compression to release the disinfecting agent onto the injection membrane 119 and around the female luer threads 120.

FIG. 3 is a prospective view of an embodiment of the capping device 100 attached to a medical implement in the open position. The second portion 108 may be shaped in such a way that it fully encircles the access portion of the needleless hub connector where the female luer threads 120 and the injection membrane 119 are located. Pressing down on the handle 111 of the second portion 108 causes the disinfecting member 116 to be displaced laterally thereby creating a gap and exposing the access portion of the medical implement for injection. This allows the syringe to connect with the access portion.

The first portion 101 may include a base 107 having the bottom opening 115 and a side defined by a side panel 102 and a top panel 103 which is angled to the base 107, wherein the side comprises a medial opening configured to face the medical implement. The side in turn is configured to extend above the access portion of the medical implement and may be permanently attached to the base at an angle of about 90°.

Protruding through the bottom opening 115 is the access portion of the medical implement with the injection membrane 119 and the female luer threads 120. The edge 114 of the bottom opening 115 shown around the access portion serves to secure the capping device and the implement together.

The capping device may further include another optional connecting element such as an elastic band, a string, or a spring connecting the first portion 101 of the capping device and the second portion 108 thereof. This additional connecting element biases the disinfecting member 116 such that when the disinfecting member 116 is displaced from the closed position, wherein the disinfecting member contacts the access portion of the medical implement, to an open position, the disinfecting member 116 is urged to return to the access portion to contact and disinfect it. This optional element may not be necessary when the connector 110 biases the second portion 108 to return medially (centrally) after the user releases the handle 111. The use of the connector 110 and the optional connecting element described above will assure 100% mandatory compliance of the medical professional with sterile techniques.

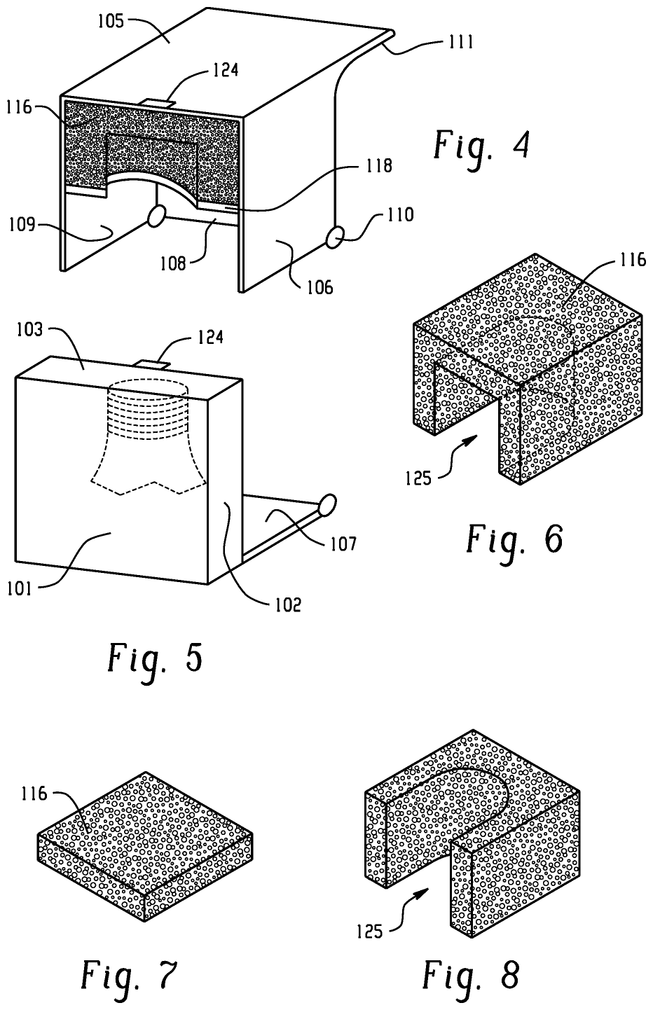

FIG. 4 is a prospective view of the second portion 108 of the capping device 100 that is detached from the medical implement. The figure shows the disinfecting member 116 and an elevated rim 118 of the sealing member 117 that supports the disinfecting member 116. The disinfecting member is located inside the second portion 108 and is affixed to the inner surface of the top chamber of the second portion. The disinfecting member 116 does not completely fill out the entire top disinfecting chamber leaving a hollow 125 (central accepting chamber) which substantially matches the size and shape of the access portion of the medical implement. The compatibility of the hollow 125 and the access portion guarantees that the disinfecting member comes into close contact with the injection membrane 119 and the female luer threads 120 of the medical implement. In an embodiment, the disinfecting member 116 nearly completely encircles the female luer threads 120, except for the part facing the first inner surface 104 of the first portion 101. Thus, for the most part, the luer threads are cleaned by the disinfecting member 116, and the remaining part of the female luer threads 120 facing the inner surface of the first portion 101 is cleaned by the disinfecting agent released from the top of the disinfecting member 116.

FIG. 4 also shows a top panel 105 of the second portion 108 having the handle 111, a lateral panel 106, and the connector 110. Inside the second portion 108 beneath the sealing member 117, the inner surface 109 of the second portion 108 is partially seen. The figure also shows a portion of an optional lock 124 located at the center of the top panel 105 which corresponds in shape and size to the other portion of the optional lock 124 present at a top panel 103 of the first portion 101 of the capping device. After injection, the medical professional may push the second portion 108 of the device centrally towards the first portion 101 to close and lock the device. The two portions of the lock would securely close the capping device. The optional lock 124 may not be necessary if the connector 110 is biased to return the second portion 108 to the first portion 101 to close the device completely and securely.

FIG. 5 is a perspective view of the first portion 101 of the capping device that is attached to the medical implement showing the side panel 102, the top panel 103, and the base 107. The figure also shows the access portion of the medical implement protruding through the bottom opening in the base 107 and having the injection membrane 119 and the female luer threads 120.

FIG. 6 is a prospective view of the disinfecting pad, which is designed to completely cover the injection membrane 119 of the medical implement, and to almost completely encircle and come in contact with the female luer threads 120. While some existing devices may only disinfect the injection membrane, the disinfecting member 116, according to the embodiment, keeps most of the female luer threads 120 completely covered. This ensures better disinfection results as the male luer threads on a syringe are often contaminated with bacteria or blood. After connection of the male luer threads from the syringe with the female luer threads from the implement, the blood or bacteria may stay on the female luer threads, and from there, spread the contamination to the injection membrane. The present device completely prevents this undesired and hazardous process. The disinfecting member 116 has the hollow 125, which substantially matches the size and shape of the access portion of the medical implement. Such compatibility guarantees that the disinfecting member 116 comes into contact with the injection membrane 119 and most of the female luer threads 120 of the medical implement.

The disinfecting member may have great absorption capacity, so when pressure is applied to push the second portion of the capping device to cover the access portion of the medical implement, compression of the disinfecting pad results in release of the disinfecting agent onto the injection membrane and around the female luer threads. This is especially important for a small portion of the female luer threads facing the first chamber as the disinfecting member 116 does not contact them directly. This small portion of the female luer threads relies on cleaning by a passive flow of the disinfecting agent in between those threads.

FIG. 7 shows top portion of the disinfecting member 116 which covers and cleans the injection membrane 119 of the medical implement. The disinfecting member 116 is a single unit and the top portion thereof is shown separately for illustrative purposes.

FIG. 8 shows the body of the disinfecting member 116 with the hollow 125. The shape and size of the chamber matches those of the medical implement except for a small medial area facing the inner surface, where the accepting chamber does not fully surround the medical implement, but leaves an open area. The disinfecting member 116 may be made of an absorbing material such as sponge. The disinfecting agent 116 may include an antibacterial agent, an antiviral agent, or a combination thereof. The disinfecting member 116 may be made of a non-woven material including polyester, silicone, cotton, polyurethane, or any other suitable material. The amount of the material for the disinfecting member 116 may vary and may be selected by a person of ordinary skill in the art. The disinfecting member 116 may be impregnated with a disinfecting agent, which is known to possess antiseptic, antibacterial, and antiviral properties, for example, povidone iodine, alcohol, chlorhexidine, or any combination thereof.

To operate the device, a medical professional may hold the medical implement with the attached capping device in one hand and may use his or her thumb to push down the second portion 108 to expose the access portion of the implement for an injection. The medical professional may hold the handle 111 down until the injection is complete. The provider may use his or her other hand to hold the syringe, connect it to the access portion of the medical implement and inject medication through the exposed hub. After the injection, the medical professional may just relive the thumb from the handle 111 and the second portion 108 would move back to close the device.

The capping device, according to an embodiment, provides a simple and affordable way to bring compliance of medical professionals to 100%. It would really take a very irresponsible healthcare professional to intentionally not close the access portion of the medical implement. With optional band/string or hinge mechanism, 100% compliance would be guaranteed. Most of the known hub protective caps Like SwabCap by ICU Medical or Curos by 3M require using two hands to attach or detach the cap from the hub. Once separated, the cap can be lost or placed on a contaminated surface. The medical professional may forget to put it back, place dirty cap back onto the hub or need to use a new clean cap. The present device is designed to avoid the above shortcomings.

The present inventive concept has been described in terms of exemplary principles and embodiments, but those skilled in the art will recognize that variations may be made and equivalents substituted for what is described without departing from the scope and spirit of the disclosure as defined by the following claims.

What is claimed is:

1. A capping device for disinfecting a medical implement, comprising:
a first portion comprising a bottom opening and a first inner surface,
a second portion comprising a medial opening, a second inner surface, and a disinfecting member attached to the second inner surface, and
a connector coupling the first portion of the capping device and the second portion thereof in a manner which permits movement of the second portion between
a closed position wherein the second portion of the capping device is in contact with the first portion thereof so as to form a hollow defined by the first inner surface and the second inner surface, and
an open position wherein the second portion is apart from the first portion,
wherein the capping device is configured for attachment to the medical implement through the bottom opening to bring an access portion of the medical implement in contact with the disinfecting member when the capping device is in the closed position.

2. The capping device according to claim 1, wherein the first portion comprises a base comprising the bottom opening and a side angled to the base, wherein the side comprises a medial opening.

3. The capping device according to claim 2, wherein the side is permanently attached to the base at an angle of about 90°.

4. The capping device according to claim 2, wherein the side is configured to extend above the access portion of the medical implement.

5. The capping device according to claim 2, wherein the side is permanently attached to base, wherein the medial opening of the side is configured to face the medical implement.

6. The capping device according to claim 1, wherein the medial opening of the second portion is configured to face the medical implement.

7. The capping device according to claim 1, wherein the second portion comprises a sealing member separating the medial opening of the second portion into a top chamber and a bottom chamber.

8. The capping device according to claim 7, wherein the disinfecting member is disposed in the top chamber of the second portion of the capping device.

9. The device according to claim 7, wherein the sealing member comprises an elevated central rim.

10. The device according to claim 7, wherein the disinfecting member is disposed over an entire inner surface of the top chamber.

11. The device according to claim 7, wherein the top chamber further comprises a hollow disposed between the disinfecting member and the medial opening, wherein the shape of the hollow substantially matches the shape of a portion of the medical implement to be disinfected.

12. The device according to claim 1, wherein the second portion further comprises a handle disposed on an external surface thereof, and wherein the second portion separates from the first portion by application of a force to the handle.

13. The device according to claim 1, wherein the disinfecting member is impregnated with a disinfecting agent.

14. The device according to claim 13, wherein when the disinfecting member is in contact with the access portion of the medical implement, the disinfecting member releases the disinfecting agent onto the portion to be disinfected.

15. The device according to claim 1, wherein the medical implement is a needleless hub or an injection port.

16. The device according to claim 1, wherein the access portion of the medical implement comprises a female luer fitting and an injection membrane.

17. The device according to claim 1, wherein the access portion of the medical implement in the open position is completely uncovered.

18. The device according to claim 1, wherein the access portion of the medical implement in the closed position may be completely covered with a disinfecting member.

19. A method for disinfecting a medical implement, comprising:
providing a device comprising:
a first portion comprising a bottom opening and a first inner surface,
a second portion comprising a medial opening, a second inner surface, and a disinfecting member attached to the second inner surface, and
a connector coupling the first portion of the capping device and the second portion thereof in a manner which permits movement of the second portion between
a closed position wherein the second portion of the capping device is in contact with the first portion thereof so as to form a hollow defined by the first inner surface and the second inner surface of the second portion, and
an open position wherein the second portion is apart from the first portion, attaching the device to an access portion of the medical implement through the bottom opening to bring the disinfecting member in contact with the access portion;
displacing the second portion from the closed position to the open position to expose the access portion of the medical implement; and
attaching a source of fluid to the medical implement.

20. The method according to claim 19, further comprising:
injecting the fluid into the medical implement;
detaching the source of fluid from the medical implement; and
displacing the second portion from the open position to the closed position to bring the disinfecting member back in contact with the portion to be disinfected.
